(19) United States
(12) Patent Application Publication
(10) Pub. No.: US 2025/0204797 A1
(43) Pub. Date: Jun. 26, 2025

(54) BLOOD PRESSURE MEASUREMENT DEVICE, CONTROL METHOD, AND CONTROL RECORDING MEDIUM

(71) Applicant: OMRON HEALTHCARE Co., Ltd., Kyoto (JP)

(72) Inventors: Nozomi NAKAWAKI, Kyoto (JP); Nobuo KUBO, Kyoto (JP); Yasunori FUKUDA, Kyoto (JP); Kazuki KUMAGAI, Kyoto (JP); Yuya HIGUCHI, Kyoto (JP)

(21) Appl. No.: 19/075,367

(22) Filed: Mar. 10, 2025

Related U.S. Application Data
(63) Continuation of application No. PCT/JP2023/040249, filed on Nov. 8, 2023.

(30) Foreign Application Priority Data
Mar. 10, 2023 (JP) 2023-037507

(51) Int. Cl. A61B 5/021; A61B 5/00; A61B 5/022; A61B 5/0225

(52) U.S. Cl. CPC A61B 5/02141; A61B 5/02225; A61B 5/0225; A61B 5/7264; A61B 2560/045; A61B 2560/0462

(57) ABSTRACT

A blood pressure measurement device that enables checking of measurement information on an information terminal in a short time, and a control method and a control recording medium for the blood pressure measurement device. A blood pressure measurement device includes a communication unit, a processor, a pressure sensor, and an adjustment mechanism that adjusts the pressure of a cuff. The processor performs measurement processing including main processing of acquiring an output of the pressure sensor while changing the pressure of the cuff by controlling the adjustment mechanism, and deriving blood pressure information based on the output, and post-processing of decreasing the pressure of the cuff to a threshold or less by controlling the adjustment mechanism, performs transmission processing of establishing a connection of near-field wireless communication with a smartphone, and transmitting information obtained in the main processing from the communication unit to the smartphone.

### CROSS-REFERENCE TO RELATED APPLICATIONS

This application is the U.S. national stage application filed pursuant to 35 U.S.C. 365(c) and 120 as a continuation of International Patent Application No. PCT/JP2023/040249, filed Nov. 8, 2023, which application claims priority to Japanese Patent Application No. 2023-037507, filed Mar. 10, 2023, which applications are incorporated herein by reference in their entireties.

### TECHNICAL FIELD

The present invention relates to a blood pressure measurement device, a control method, and a control recording medium.

### BACKGROUND ART

When information measured by a blood pressure measurement device is recorded and analyzed, it is desirable that the blood pressure measurement device and an information terminal are communicably connected to each other so that the information terminal can automatically acquire the measured information, instead of a user inputting the measured information to the information terminal each time. Specifically, for example, a method in which the information terminal receives the measured information from the blood pressure measurement device by near-field wireless communication such as Bluetooth (registered trademark) is conceivable.

Patent Literature 1 discloses a wearable terminal including a biological information measurement unit and a wireless communication unit. The wireless communication unit transmits, to a disease onset risk prediction device via a wireless communication network, biological information including blood pressure data and data indicating an occurrence status of irregular pulse waves that are generated by the biological information measurement unit and the measurement time thereof, each time the biological information is generated or after the biological information is accumulated for a certain period.

### CITATION LIST

### Patent Literature

Patent Literature 1: JP 2019-115614 A

### SUMMARY OF INVENTION

### Technical Problem

When the amount of information transmitted from the blood pressure measurement device to the information terminal increases, it takes a long time to transmit the information. When the transmission of the information takes a long time, for example, although the blood pressure measurement device has completed the measurement and displays blood pressure information, the measurement result may not be checked for a while on the information terminal connected to the blood pressure measurement device. Even in a case where the amount of information transmitted from the blood pressure measurement device to the information terminal is small, when it takes a long time to transmit the information, the measurement result may not be checked on the information terminal for a while.

An object of the present invention is to provide a blood pressure measurement device that enables checking of measurement information on an information terminal in a short time, and provide a control method and a control recording medium.

### Solution to Problem

The technique of the present disclosure is as follows. Note that components and the like according to the following embodiments are indicated in parentheses, but the components are not limited thereto.

(1) A blood pressure measurement device (a blood pressure measurement device 100) including:
a near-field wireless communication unit (a communication unit 15);
a processor (a processor 11);
a measurement sensor (a pressure sensor 32); and
an adjustment mechanism (an adjustment mechanism 50) configured to adjust a pressure of a cuff (a cuff 20), wherein
the processor is configured to
perform measurement processing including first processing (main processing) of acquiring an output of the measurement sensor while changing the pressure of the cuff by controlling the adjustment mechanism, and deriving blood pressure information (a systolic blood pressure, a diastolic blood pressure, a mean blood pressure value, a pulse, an augmentation index (AI) value, or the like) based on the output, and second processing (post-processing) of decreasing the pressure of the cuff to a threshold or less by controlling the adjustment mechanism,
perform transmission processing of establishing a connection of near-field wireless communication with an information terminal (a smartphone 200), and transmitting information obtained in the first processing (the blood pressure information or pulse wave data obtained in a course of deriving the blood pressure information) from the near-field wireless communication unit to the information terminal, and
start the transmission processing at a timing (a time T3) before an end timing (a time T4) of the measurement processing.

According to (1), since the transmission processing is started before the measurement processing is ended, for example, it is possible to shorten the time until the transmission of the information obtained in the first processing to the information terminal is completed as compared to a case where the transmission processing is started after the measurement processing is ended. Thus, the blood pressure information or the like can be checked on the information terminal in a short time after the measurement processing is completed, and user satisfaction can be increased.

(2) The blood pressure measurement device according to (1), wherein the processor starts the transmission processing during a period from an end of the first processing to an end of the second processing (between the time T3 and the time T4).

According to (2), since the transmission processing is not started during a period in which the first processing, including processing (pulse wave data acquisition processing) that may affect the accuracy in measuring the blood pressure information, is performed, it is possible to prevent the transmission processing from affecting the accuracy in measuring the blood pressure information and to improve the accuracy in measuring the blood pressure information.

(3) The blood pressure measurement device according to (2), wherein the processor starts the transmission processing at a timing (a time T3) at which the first processing is ended.

According to (3), the information can be transmitted to the information terminal earlier.

(4) The blood pressure measurement device according to (1), wherein the processor starts the transmission processing when the output of the measurement sensor acquired in the first processing satisfies a condition necessary for deriving the blood pressure information.

According to (4), since the transmission processing is started in a state in which the condition necessary for deriving the blood pressure information is satisfied, it is possible to prevent the transmission processing from affecting the accuracy in measuring the blood pressure information, and to improve the accuracy in measuring the blood pressure information. In addition, since the transmission processing is started during the first processing, it is possible to further shorten the time until the transmission of the information is completed.

(5) The blood pressure measurement device according to (4), wherein the processor starts the transmission processing during a period between a timing (a time T2) at which the condition is satisfied and a timing (the time T3) at which derivation of the blood pressure information is completed, in the first processing.

According to (5), since the transmission processing is started in a state in which the condition necessary for deriving the blood pressure information is satisfied, it is possible to prevent the transmission processing from affecting the accuracy in measuring the blood pressure information, and to improve the accuracy in measuring the blood pressure information. In addition, since the transmission processing is started during the first processing, it is possible to further shorten the time until the transmission of the information is completed.

(6) The blood pressure measurement device according to (5), wherein the processor starts the transmission processing at a timing (a time T2) at which the condition is satisfied in the first processing.

According to (6), it is possible to further shorten the time until the transmission of the information is completed.

(7) A control method for a blood pressure measurement device including a near-field wireless communication unit, a measurement sensor, and an adjustment mechanism configured to adjust a pressure of a cuff,
the control method causing a processor to execute steps of:
performing measurement processing including first processing of acquiring an output of the measurement sensor while changing the pressure of the cuff by controlling the adjustment mechanism, and deriving blood pressure information based on the output, and second processing of decreasing the pressure of the cuff to a threshold or less by controlling the adjustment mechanism;
performing transmission processing of establishing a connection of near-field wireless communication with an information terminal, and transmitting information obtained in the first processing from the near-field wireless communication unit to the information terminal; and
starting the transmission processing at a timing before an end timing of the measurement processing.

(8) A control recording medium for a blood pressure measurement device including a near-field wireless communication unit, a measurement sensor, and an adjustment mechanism configured to adjust a pressure of a cuff,
the control recording medium causing a processor to execute steps of.
performing measurement processing including first processing of acquiring an output of the measurement sensor while changing the pressure of the cuff by controlling the adjustment mechanism, and deriving blood pressure information based on the output, and second processing of decreasing the pressure of the cuff to a threshold or less by controlling the adjustment mechanism;
performing transmission processing of establishing a connection of near-field wireless communication with an information terminal, and transmitting information obtained in the first processing from the near-field wireless communication unit to the information terminal; and
starting the transmission processing at a timing before an end timing of the measurement processing.

### Advantageous Effects of Invention

According to the present invention, a measurement result can be checked on an information terminal in a short time.

### BRIEF DESCRIPTION OF DRAWINGS

FIG. 1 is a schematic diagram illustrating a schematic configuration of a management system.

FIG. 2 is a timing chart for explaining a processing content of a processor 11 during measurement of blood pressure information.

FIG. 3 is a timing chart for explaining a modified example of the processing content of the processor 11 during measurement of blood pressure information.

### DESCRIPTION OF EMBODIMENTS

### Overview of Blood Pressure Measurement Device and Information Terminal of the Present Disclosure

A blood pressure measurement device and an information terminal according to the present disclosure can communicate with each other by near-field wireless communication. When an operation for measuring blood pressure information is detected, a processor of the blood pressure measurement device including an adjustment mechanism adjusting the pressure of a cuff and a measurement sensor performs measurement processing including first processing of acquiring an output of the measurement sensor while changing the pressure of the cuff by controlling the adjustment mechanism, and deriving blood pressure information based on the output, and second processing of decreasing the pressure of the cuff to a threshold or less by controlling the adjustment mechanism, and performs transmission processing of establishing a connection of near-field wireless communication with an information terminal, and transmitting information obtained in the first processing from a near-field wireless communication unit to the information terminal. The processor starts the transmission processing at a timing before an end timing of the measurement processing. Accordingly, since the transmission processing is started before the measurement processing is ended, for example, it is possible to shorten the time until the transmission of the information obtained in the first processing to the information terminal is completed as compared to a case where the transmission processing is started after the measurement processing is ended, and to increase user satisfaction.

Hereinafter, a configuration example of a management system including the blood pressure measurement device and the information terminal of the present disclosure will be described.

### System Configuration

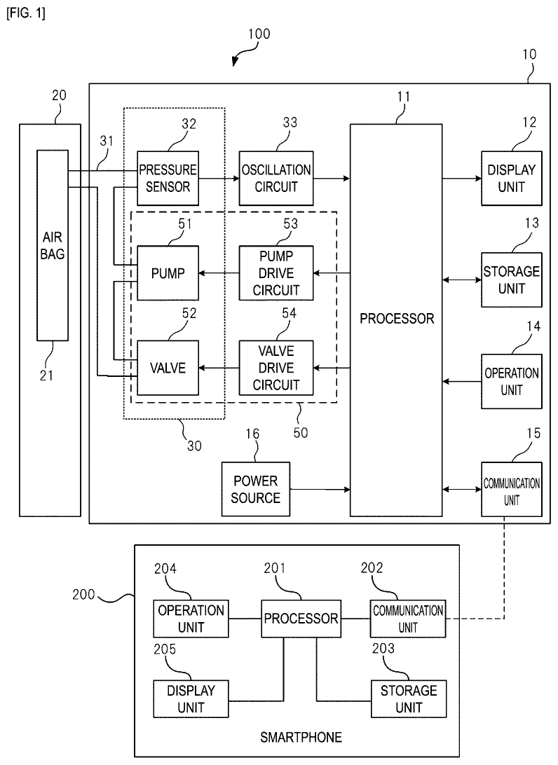

FIG. 1 is a schematic diagram illustrating a schematic configuration of the management system. The management system includes a blood pressure measurement device 100 and a smartphone 200 as an example of the information terminal. The blood pressure measurement device 100 and the smartphone 200 are configured to be communicable with each other by near-field wireless communication. The information terminal is not limited to a smartphone, and may be a personal computer, a tablet terminal, or the like. A method of the near-field wireless communication is not particularly limited, and for example, a method such as Wi-Fi (registered trademark), ANT, Bluetooth (registered trademark), or infrared communication can be used. In the following description, it is assumed that the near-field wireless communication method is Bluetooth (registered trademark). Hereinafter, an owner who owns both the blood pressure measurement device 100 and the smartphone 200 is referred to as a user.

### Blood Pressure Measurement Device

The blood pressure measurement device 100 measures the blood pressure information of the user (a person to be measured) and includes a main body portion 10, a cuff 20 that can be wrapped around a measurement site (e.g., an upper arm) of the user, and an air tube 31 that couples the main body portion 10 and the cuff 20. The “blood pressure information” is information indicating characteristics of a circulatory system and includes a pulse wave and indices that can be calculated from the pulse wave, such as a systolic blood pressure, a diastolic blood pressure, a mean blood pressure value, a pulse, and an augmentation index (AI) value, for example. In the following description, it is assumed that the blood pressure information corresponds to a systolic blood pressure and a diastolic blood pressure.

The main body portion 10 includes an air system 30 connected to the air tube 31, an oscillation circuit 33 connected to the air system 30, an adjustment mechanism 50 that adjusts the pressure of the cuff 20, a processor 11, a display unit 12, a storage unit 13, an operation unit 14, a communication unit 15, and a power source 16.

The air system 30 includes a pressure sensor 32 for detecting the pressure (cuff pressure) in an air bag 21 provided at the cuff 20, a pump 51 for supplying air to the air bag 21 in order to increase the cuff pressure, and a valve 52 that is opened and closed in order to discharge or enclose the air in the airbag 21. The pressure sensor 32 is one of measurement sensors used to measure the blood pressure information.

The pressure sensor 32 is, for example, a capacitance pressure sensor, and the capacitance value thereof changes in accordance with the cuff pressure. The oscillation circuit 33 is connected to the pressure sensor 32 and outputs a signal of an oscillation frequency corresponding to the capacitance value of the pressure sensor 32 to the processor 11. The processor 11 converts the signal obtained from the oscillation circuit 33 into a pressure to detect the cuff pressure.

The adjustment mechanism 50 is a mechanism that adjusts the cuff pressure based on a control signal provided from the processor 11. The adjustment mechanism 50 includes a pump drive circuit 53 and a valve drive circuit 54 in addition to the pump 51 and the valve 52 which are parts of the air system 30. The pump drive circuit 53 drives the pump 51 based on a control signal provided from the processor 11. The valve drive circuit 54 controls opening and closing of the valve 52 based on a control signal provided from the processor 11. The adjustment mechanism 50 is not limited to the illustrated configuration.

The display unit 12 includes, for example, an organic electro-luminescence (EL) display, a liquid crystal display, or the like, and displays the measured blood pressure information and the like.

The storage unit 13 includes a non-transitory storage medium such as a flash memory in addition to a working memory such as a random access memory (RAM).

Various types of information such as measured blood pressure information are stored in this storage medium. A control recording medium to be executed by the processor 11 is also stored in this storage medium.

The operation unit 14 is an input unit such as a button or a touch panel that receives an input from the user, and receives various operations such as ON/OFF of a power source, start of measurement of the blood pressure information, and selection of an item from the user. The operation unit 14 includes, for example, a measurement start button used to instruct the start of measurement of the blood pressure information.

The communication unit 15 is a communication interface for performing near-field wireless communication, and includes a communication antenna and various circuits.

The processor 11 comprehensively controls each unit of the main body portion 10. The processor 11 is, for example, a central processing unit (CPU) that is a general-purpose processor executing software (recording medium) to perform various functions, a programmable logic device (PLD) that is a processor whose circuit configuration can be changed after manufacturing, such as a field programmable gate array (FPGA), or a dedicated electric circuit that is a processor having a circuit configuration dedicatedly designed to execute specific processing, such as an application specific integrated circuit (ASIC). The processor 11 may be configured with one processor, or may be configured with a combination of two or more processors of the same type or different types (for example, a plurality of FPGAs or a combination of a CPU and an FPGA). More specifically, the hardware structure of the processor 11 is an electric circuit (circuitry) in which circuit elements such as semiconductor elements are combined.

Upon detecting the pressing of the measurement start button included in the operation unit 14, the processor 11 receives an instruction to start measurement, pressurizes the cuff 20, and derives blood pressure information based on an output of the pressure sensor 32 obtained at an appropriate cuff pressure. The processor 11 displays the derived blood pressure information on the display unit 12. The processor 11 transmits measurement data including the derived blood pressure information and other information such as a pulse wave amplitude obtained in the course of deriving the blood pressure information from the communication unit 15 to the smartphone 200.

### Information Terminal

The smartphone 200 includes a processor 201, a communication unit 202, a storage unit 203, an operation unit 204, and a display unit 205.

The communication unit 202 is a communication interface for performing near-field wireless communication, and includes a communication antenna and various circuits.

The storage unit 203 includes a non-transitory storage medium such as a flash memory in addition to a working memory such as a RAM.

The operation unit 204 is an input unit such as a button or a touch panel that receives an input from the user, and receives various operations from the user.

The display unit 205 includes, for example, an organic EL display or a liquid crystal display.

The processor 201 comprehensively controls each unit of the smartphone 200. Upon receiving measurement data from the blood pressure measurement device 100 via the communication unit 202, the processor 201 stores the measurement data in the storage unit 203. The processor 201 performs control of displaying the measurement data stored in the storage unit 203 on the display unit 205. As a result, the user can check the details of the measurement data obtained through the measurement with the blood pressure measurement device 100 on the smartphone 200, and check a time-series change or the like of the measurement data of a plurality of times of measurements on the smartphone 200.

### Method for Measuring Blood Pressure Information

The processor 11 of the blood pressure measurement device 100 calculates blood pressure information in accordance with an oscillometric method. Upon detecting the pressing of the measurement start button, the processor 11 controls the adjustment mechanism 50 to perform pressurization control of increasing the cuff pressure from the initial state to a predetermined value. After the cuff pressure is increased to the predetermined value which is high enough to be equal to or higher than the systolic blood pressure of the user, the processor 11 performs depressurization control of decreasing the cuff pressure at a constant rate. The processor 11 acquires an output signal of the oscillation circuit 33 and converts the output signal into a pressure value to detect a cuff pressure during a period in which the depressurization control is performed.

The processor 11 detects, from the detected cuff pressure, a steady pressure (a compression pressure applied to the measurement site by the cuff 20) and pulse wave data which is a pressure component superimposed on the compression pressure in synchronization with the pulse of the living body by, for example, filter processing. The processor 11 derives an amplitude value of the detected pulse wave data (hereinafter referred to as a pulse wave amplitude), and determines the systolic blood pressure and the diastolic blood pressure by a known method using data (pulse wave envelope) indicating a relationship between the derived pulse wave amplitude and the compression pressure when the pulse wave data is obtained. The processor 11 can also detect a cuff pressure and generate a pulse wave envelope during the pressurization control. According to the oscillometric method, it can be said that the accuracy in calculating a blood pressure value depends on the shape of the pulse wave envelope.

In the blood pressure measurement device 100 of the present embodiment, the processor 11 handles information including at least one of blood pressure information (a systolic blood pressure and a diastolic blood pressure) derived based on a cuff pressure detected during the depressurization control and other information such as pulse wave data obtained in the course of deriving the blood pressure information as measurement data, and performs control of transmitting the measurement data to the smartphone 200. In transmitting the measurement data by near-field wireless communication, it is important to prevent noises due to the influence of radio waves or the like from being included in the output of the pressure sensor 32 and the output of the oscillation circuit 33 in order to improve the accuracy in deriving the blood pressure information. In the present embodiment, the processor 11 appropriately controls the transmission timing of the measurement data, so that the accuracy in deriving the blood pressure information is not affected. Hereinafter, details of the operation during measurement of the blood pressure information will be described.

### Measurement Operation

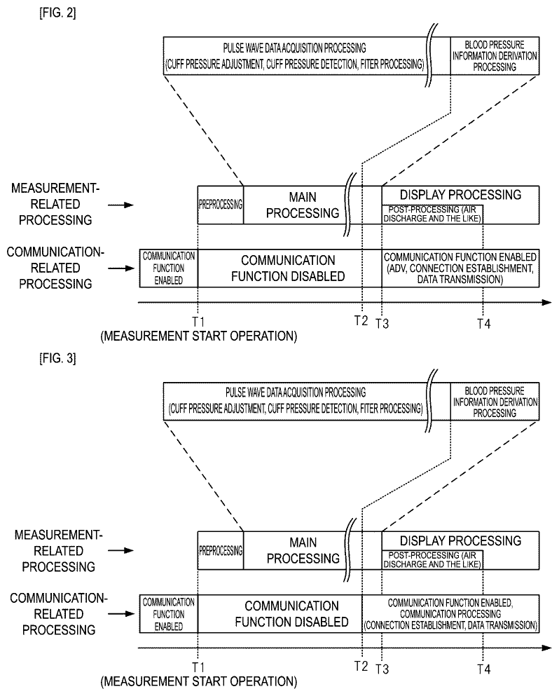

FIG. 2 is a timing chart for explaining a processing content of the processor 11 during measurement of the blood pressure information. FIG. 2 illustrates measurement-related processing related to the measurement of the blood pressure information and communication-related processing related to communication performed by the communication unit 15.

Upon detecting a power-on operation of the main body portion 10, the processor 11 enables a communication function of the communication unit 15. The communication function includes transmitting an advertisement signal from the communication unit 15, establishing a connection of the communication unit 15 with an external device responding to the advertisement signal, pairing with the external device, transmitting and receiving data to and from the external device, and the like. There may be a configuration in which the communication function is enabled even when the power source of the main body portion 10 is off. Examples of a method for disabling the communication function include a method of stopping power supply to the communication unit 15 and a method of bringing the communication unit 15 into a pause state while power supply is performed.

Upon detecting the pressing of the measurement start button at a time T1, the processor 11 disables the communication function of the communication unit 15. In addition, the processor 11 starts preprocessing for the measurement of the blood pressure information. The preprocessing includes, for example, initializing a predetermined region of the storage unit 13, discharging air from the air bag 21, or correcting the pressure sensor 32.

Upon completion of the preprocessing, the processor 11 performs main processing. The main processing includes pulse wave data acquisition processing and blood pressure information derivation processing. The pulse wave data acquisition processing includes cuff pressure adjustment processing of performing the pressurization control and the depressurization control described above, and data acquisition processing of processing the cuff pressure detected during the depressurization control to acquire a compression pressure, pulse wave data, and a pulse wave amplitude. The pulse wave data acquisition processing can be ended at a time point when a condition necessary for deriving the blood pressure information is satisfied. This condition is that the required number of pulse wave amplitudes (or detected cuff pressures for acquiring the pulse wave amplitudes) for determining a systolic blood pressure and a diastolic blood pressure are obtained.

The blood pressure information derivation processing is processing of determining a systolic blood pressure and a diastolic blood pressure based on a pulse wave envelope indicating a relationship between the pulse wave amplitude and the compression pressure obtained in the data acquisition processing. In the example of FIG. 2, after the preprocessing is ended, the pulse wave data acquisition processing is ended at a time T2, and then the blood pressure information derivation processing is ended at a time T3, so that the systolic blood pressure and the diastolic blood pressure are determined.

When the blood pressure information derivation processing is ended at the time T3, the processor 11 performs display processing of displaying the determined systolic blood pressure and diastolic blood pressure on the display unit 12, and performs the post-processing in parallel with the display processing. Specifically, the post-processing includes air discharge processing of decreasing the pressure of the cuff 20 to a threshold or less by controlling the adjustment mechanism 50. The threshold is, for example, an atmospheric pressure. That is, the air discharge processing is processing of discharging most of the air injected in the air bag 21 to the outside so that the cuff 20 can be detached from the measurement site.

This post-processing can also be started during a period in which the blood pressure information derivation processing is performed. That is, the start timing of the post-processing may be between the time T2 and the time T3. In the example of FIG. 2, the post-processing is started at the time T3, and the post-processing is ended at the time T4.

The measurement processing of measuring the blood pressure information is made up of the preprocessing, the main processing, and the post-processing described above. The main processing constitutes the first processing, and the post-processing constitutes the second processing.

When the blood pressure information derivation processing is ended at the time T3, the processor 11 enables the communication function and establishes a connection between the communication unit 15 and the smartphone 200. Then, the processor 11 performs transmission processing of transmitting measurement data including at least one of the blood pressure information including the systolic blood pressure and the diastolic blood pressure determined in the blood pressure information derivation processing and the pulse wave data obtained in the course of deriving the blood pressure information from the communication unit 15 to the smartphone 200. As described above, the establishment of a connection with the smartphone 200 and the subsequent transmission of the measurement data to the smartphone 200 are performed in parallel with the post-processing.

### Effect of Embodiment

According to the blood pressure measurement device 100, preparation for transmission of measurement data and transmission of the measurement data can be performed while the post-processing is performed. Thus, the timing at which transfer of the measurement data to the smartphone 200 is completed can be advanced as compared to a configuration in which preparation for transmission of measurement data and transmission of the measurement data are enabled after the post-processing. After the post-processing is ended and the cuff 20 is removed from the measurement site, the user can check the details of the measurement data on the smartphone 200 without waiting for a long time. As a result, user satisfaction can be increased.

### Modified Example

The timing at which the communication function is enabled for the first time after the pressing of the measurement start button is detected may be the time T2 as illustrated in FIG. 3. This is because, since data of pulse wave envelope required to determine the blood pressure information has been acquired at the time T2, it is possible to prevent the accuracy in deriving the blood pressure information from being affected even when the communication function is enabled at this timing at the time T2. When the communication function is enabled at the time T2, the timing at which transfer of the measurement date to the smartphone 200 is completed can be further advanced, which is preferable.

The timing at which the communication function is enabled is not limited to the time T2, and may be any timing between the time T2 and the time T3. Even in this case, the timing at which transfer of the measurement data to the smartphone 200 is completed can be advanced as compared to the operation in FIG. 2.

Note that the communication function may be enabled at a timing after a short time has elapsed since the start of the post-processing. For example, the communication function may be enabled between the time T3 and the time T4. Even in this case, the timing at which transfer of the measurement data to the smartphone 200 is completed can be advanced as compared with a case where the communication function is enabled after the end of the post-processing. In addition, since the air in the air bag 21 is rapidly discharged immediately after the start of the post-processing, when the communication function is enabled at a timing at which the air is discharged to some extent, the communication can be performed satisfactorily. In the operation of FIG. 2, when the start timing of the post-processing is set between the time T3 and the time T4, the communication can be prevented from being started immediately after the start of the post-processing.

The example of deriving the blood pressure information by the oscillometric method has been described above, but the processor 11 may detect a Korotkoff sound and derive the blood pressure information based on the detected Korotkoff sound. In that case, a microphone is provided as the measurement sensor instead of the pressure sensor 32 and the oscillation circuit 33. The processor 11 acquires an amplitude value of a sound detected by the microphone during the depressurization control instead of a pulse wave amplitude value, and derives the blood pressure information based on the amplitude value.

Although various embodiments have been described above, it is needless to say that the present invention is not limited to these examples. It will be apparent to those skilled in the art that various changes and modifications can be made within the scope of the claims, and it is understood that these naturally fall within the technical scope of the present invention. Further, components of the above-described embodiments may be combined as desired without departing from the gist of the present invention.

Note that the present application is based on Japanese Patent Application filed on Mar. 10, 2023 (JP 2023-037507), the content of which is incorporated herein by reference.

### REFERENCE NUMERALS LIST

10 Main body portion
100 Blood pressure measurement device
11, 201 Processor
12, 205 Display unit
13, 203 Storage unit
14, 204 Operation unit
15, 202 Communication unit
16 Power source
20 Cuff
21 Air bag
30 Air system
31 Air tube
32 Pressure sensor
33 Oscillation circuit
50 Adjustment mechanism
51 Pump
52 Valve
53 Pump drive circuit
54 Valve drive circuit
200 Smartphone

1. A blood pressure measurement device comprising:
a near-field wireless communication unit;
a processor;
a measurement sensor; and,
an adjustment mechanism configured to adjust a pressure of a cuff,
wherein the processor is configured to:
perform measurement processing including first processing of acquiring an output of the measurement sensor while changing the pressure of the cuff by controlling the adjustment mechanism, and deriving blood pressure information based on the output, and second processing of decreasing the pressure of the cuff to a threshold or less by controlling the adjustment mechanism,
perform transmission processing of establishing a connection of near-field wireless communication with an information terminal, and transmitting information obtained in the first processing from the near-field wireless communication unit to the information terminal,
start the transmission processing at a timing before an end timing of the measurement processing, and
start the transmission processing when the output of the measurement sensor acquired in the first processing satisfies a condition necessary for deriving the blood pressure information.

2. The blood pressure measurement device according to claim 1, wherein the processor starts the transmission processing during a period from an end of the first processing to an end of the second processing.

3. The blood pressure measurement device according to claim 2, wherein the processor starts the transmission processing at a timing at which the first processing is ended.

4. The blood pressure measurement device according to claim 1, wherein the processor starts the transmission processing during a period between a timing at which the condition is satisfied and a timing at which derivation of the blood pressure information is completed, in the first processing.

5. The blood pressure measurement device according to claim 4, wherein the processor starts the transmission processing at a timing at which the condition is satisfied in the first processing.

6. A control method for a blood pressure measurement device including a near-field wireless communication unit, a measurement sensor, and an adjustment mechanism configured to adjust a pressure of a cuff,
the control method causing a processor to execute steps of:
performing measurement processing including first processing of acquiring an output of the measurement sensor while changing the pressure of the cuff by controlling the adjustment mechanism, and deriving blood pressure information based on the output, and second processing of decreasing the pressure of the cuff to a threshold or less by controlling the adjustment mechanism;
performing transmission processing of establishing a connection of near-field wireless communication with an information terminal, and transmitting information obtained in the first processing from the near-field wireless communication unit to the information terminal;
starting the transmission processing at a timing before an end timing of the measurement processing; and,
starting the transmission processing when the output of the measurement sensor acquired in the first processing satisfies a condition necessary for deriving the blood pressure information.

7. A control recording medium for a blood pressure measurement device including a near-field wireless communication unit, a measurement sensor, and an adjustment mechanism configured to adjust a pressure of a cuff,
the control recording medium causing a processor to execute steps of:
performing measurement processing including first processing of acquiring an output of the measurement sensor while changing the pressure of the cuff by controlling the adjustment mechanism, and deriving blood pressure information based on the output, and second processing of decreasing the pressure of the cuff to a threshold or less by controlling the adjustment mechanism;
performing transmission processing of establishing a connection of near-field wireless communication with an information terminal, and transmitting information obtained in the first processing from the near-field wireless communication unit to the information terminal;
starting the transmission processing at a timing before an end timing of the measurement processing; and
starting the transmission processing when the output of the measurement sensor acquired in the first processing satisfies a condition necessary for deriving the blood pressure information.
